(19) United States
(12) Patent Application Publication
(10) Pub. No.: US 2013/0052234 A1
(43) Pub. Date: Feb. 28, 2013

(54) EDIBLE STRIPS

(71) Applicants: Michael Goldberg, Englewood, NJ (US); Ehud Arbit, Englewood, NJ (US)

(72) Inventors: Michael Goldberg, Englewood, NJ (US); Ehud Arbit, Englewood, NJ (US)

(73) Assignee: Purebrands LLC, Boca Raton, FL (US)

(21) Appl. No.: 13/566,495

(22) Filed: Aug. 3, 2012

Related U.S. Application Data
(60) Provisional application No. 61/641,056, filed on May 1, 2012.
(60) Provisional application No. 61/527,351, filed on Aug. 25, 2011.

(51) Int. Cl. A61K 9/00; A61K 31/355; A61K 31/714; A61K 31/675; A61P 25/00; A61K 31/4188; A61K 31/4045; A61K 36/815; A61K 36/28; A61P 3/02; A61K 31/522; A61K 31/197

(52) U.S. Cl. 424/400; 514/263.34; 514/458; 514/52; 514/89; 514/563; 514/387; 514/263.31; 514/263.32; 514/415; 424/777; 424/764

(57) ABSTRACT

An edible oral strip composition includes a therapeutically effective amount of active agent(s) to provide at least one effect selected from a stimulating effect, an increased physical endurance, alleviate temporary fatigue, improve nervous system functions, and combinations of any of the foregoing. In additional embodiments, the edible strip composition includes a therapeutically effective amount of active agent(s) to provide sleep aid.

### FIELD OF THE INVENTION

The present invention relates to oral edible strip compositions. The oral strips preferably include one or more active agents (e.g., therapeutic agents and/or nutraceutical agents) and one or more flavoring agents, and are preferably placed in the oral cavity to release the agent from the composition.

### BACKGROUND OF THE INVENTION

Applicant hereby claims priority to U.S. Patent Application Ser. No. 61/641,056, filed on May 1, 2012, and U.S. Patent Application Ser. No. 61/527,351, filed on Aug. 25, 2011, the disclosures of which are hereby incorporated by reference into the present application.

The desire for compositions which provide quick energy boosts to individuals has driven demand for functional beverages, and drinks. A functional beverage is a beverage that not only quenches thirst, but also has added nutritional benefits. For example, sports drinks provide for rehydration and electrolyte replenishment while, vitamin-enriched water or meal replacement drinks provide a source of specific nutrition. Another category of functional beverages is energy drinks. Energy drinks are intended to produce an alert mental state in addition to a high-energy physical state. Energy drinks are particularly attractive to individuals participating in physically and mentally demanding activities in order to alleviate mental and physical fatigue.

The basis for many energy drinks is a combination of caffeine and a carbohydrate such as glucose. Two double-blind, placebo-controlled, cross-over studies demonstrated that a beverage containing glucose and caffeine can improve cognitive performance (Kennedy D O, Scholey A B. A glucose-caffeine ‘energy drink’ ameliorates subjective and performance deficits during prolonged cognitive demand. Appetite. 2004 June; 42(3):331-3). Combined glucose-caffeine drinks have also been shown to improve attention and performance on mental tasks requiring specific attention (Rao A, Hu H, Nobre A C. The effects of combined caffeine and glucose drinks on attention in the human brain. Nutr Neurosci. 2005 June; 8(3):141-53). Another study found that an energy drink including glucose and caffeine improved specific aspects of memory and attention in a synergistic way that could not be anticipated from individual components (Scholey A B, Kennedy D 0. Cognitive and physiological effects of an “energy drink”: an evaluation of the whole drink and of glucose, caffeine and herbal flavouring fractions. Psychopharmacology (Berl). 2004 November; 176(3-4):320-30).

A number of patents have attempted to provide formulations to increase mental and/or physical energy. For example, U.S. Pat. No. 6,261,589 entitled “Dietary supplement nutrient soft drink composition with psychoactive effect” purports to describe a beverage composition that is a nutrient dietary supplement for increasing energy level and general awareness. It is a carbonated beverage containing phenylalanine, Vitamin B-6, Vitamin C, copper, folic acid, taurine, Vitamin B-5 (or pro-Vitamin B-5), choline, fruit sugar, caffeine, and optionally, green tea. The composition is not purported to provide an increase in physical energy and does not contain ginseng or guarana.

U.S. Pat. No. 6,207,203 entitled “Fortified coffee drink” purports to describe a fortified coffee drink providing protein, vitamins and minerals, in addition to caffeine. This invention, devoid of ginseng or taurine, is intended to increase awareness.

U.S. Patent Application No.: 20030104107A1 entitled “Energy drink formula and method” discloses the formulation of an energy drink including the ingredients glucose, Vitamin B12 and pantothenic acid. The composition does not include caffeine, taurine or ginseng.

U.S. Patent Application No.: 20040018275A1 entitled “Carbonated energy beverage” describes a composition containing a saccharide, tea or coffee concentrate and B-Vitamin complex.

Dietary compositions such as the so-called energy drinks are typically designed to give the user a burst of energy after oral consumption of the energy drink. Some energy drink formulations include a combination of methylxanthine, B Vitamins, and exotic herbal ingredients.

Dietary compositions for aiding sleep are also known. For example, U.S. Patent Application Publication No. 2011/0081385 (Cremisi) is directed to compositions and methods for the regulation of sleep and circadian rhythms, said to contain melatonin and one or more vitamins that are said to enhance the effectiveness of melatonin. Preferred vitamins include folic acid, riboflavin (vitamin B₂), cobalamin (vitamin B₁₂) or pyridoxine (vitamin B₆).

Therapeutically active agents (e.g., drugs and/or nutraceuticals) taken by mouth and swallowed are absorbed first into the blood perfusing the gastrointestinal (GI) tract. The venous drainage from the GI tract is first passed into the blood perfusing the liver, the major detoxifying organ of the body. In addition to protecting the organism from ingested toxins, the liver also metabolizes these active agents, which may be inactivated by first pass metabolism in the liver. Blood from the liver then returns to the left side of the heart via the hepatic portal vein and reaches the rest of the systemic circulation. This first pass through the liver may result in the removal of a substantial proportion of an ingested active agent.

The above-mentioned patents and patent applications are all hereby incorporated by reference.

### SUMMARY OF THE INVENTION

It is an object of the present invention to provide an edible film composition in the form of an oral strip which can increase energy and/or mental alertness in a human.

It is an object of the present invention to provide an edible film composition in the form of an oral strip which can act as a sleep aid in a human.

It is an object of the present invention to provide an edible film composition in the form of an oral strip which has stimulating effects and increase physical endurance, alleviate temporary fatigue and/or improve nervous system functions.

It is another object of the present invention to provide a method of increasing energy and mental alertness which includes the step of administering to a human.

It is another object of the present invention to provide a process for producing an edible oral strip composition which incorporates one or more active agent(s) with a flavoring agent(s) which can increase energy and/or mental alertness in a human.

It is another object of the present invention to provide a process for producing an edible oral strip composition which incorporates one or more active agent(s) with a flavoring agent(s) which can act as a sleep aid(s) in a human.

It is a further object of the present invention to provide compositions and methods for increasing energy and/or mental alertness in a human without increasing body fluid content.

In accordance with the above objects and others, the present invention is directed, in part, to an edible oral strip composition which contains one or more active agents along with one or more flavoring agents, which composition quickly releases the active agent(s) from the strip within, e.g., about 1 minute or less, after being placed in the oral cavity of a human. The active agent(s) are included in a therapeutically effective amount such that the edible oral strip has stimulating effects and increase physical endurance, alleviate temporary fatigue and/or improve nervous system functions.

In accordance with the above objects and others, the present invention is also directed, in part, to an edible oral strip composition which contains one or more active agents along with one or more flavoring agents, which composition quickly releases the active agent(s) from the strip within, e.g., about 1 minute or less, after being placed in the oral cavity of a human. The active agent(s) are included in a therapeutically effective amount such that the edible oral strip acts as a sleep aid to a human who ingests the same.

In certain preferred embodiments, the edible oral strip of the present invention is a quick dissolving edible film.

In certain preferred embodiments directed to energy strips, the edible film composition includes caffeine as an active agent.

In certain preferred embodiments directed to sleep aid strips, the edible film composition includes melatonin as an active agent.

In certain preferred embodiments, the edible energy film composition includes a combination of Caffeine, Vitamin E and one or more B Vitamins. In certain preferred embodiments, the B Vitamins include one or more B Vitamins selected from Vitamin B6, Vitamin B12, Vitamin B5, and Vitamin B7. In certain preferred embodiments, the edible film composition includes a combination of Caffeine, Vitamin E and one or more B Vitamins which promote increased energy and/or mental alertness in a human within a short period of time after being placed in the oral cavity of a human (e.g., within about 1 to about 30 minutes).

In certain preferred embodiments, the edible sleep aid film composition includes melatonin, L-theanine, goji berry, chamomile, and/or combinations of any of the foregoing. In certain preferred embodiments, the edible film composition includes a combination of Caffeine, Vitamin E and one or more B Vitamins which promote increased energy and/or mental alertness in a human within a short period of time after being placed in the oral cavity of a human (e.g., within about 1 to about 30 minutes).

In certain embodiments, the edible film composition provided as an oral strip comprises a matrix of (i) a thermoplastic food grade material selected from, e.g., hydroxypropylmethylcellulose(s), pullulan, functional equivalents thereof, and mixtures thereof; (ii) a suitable amount of a plasticizer for the thermoplastic food grade material, e.g., glycerin; and (iii) an effective amount of a flavoring agent incorporated therein. Preferably, the oral strip has a thickness from about 2 mil to about 25 mil and releases the active agent(s) within about 1 minute, e.g., when placed in the oral cavity of a human. More preferably, the oral strip quickly releases the active agent(s) when placed in the oral cavity of a human, e.g., within about 30 seconds or less.

In certain preferred embodiments, the ingredients of the edible film composition (oral strip) of the invention are GRAS materials (“generally regarded as safe”) and/or are food grade. However, one skilled in the art will appreciate that the categorization and qualification of a given material as food grade may vary from country to country, and further that a material that has not been classified as food grade presently may indeed qualify and be classified as food grade in the future. Accordingly, it is contemplated that all such materials that are or become food grade may constitute ingredients of the edible oral strip compositions of the present invention.

In certain preferred embodiments, the edible oral strip composition may have a thickness is from about 2 mil to about 25 mil, and in certain embodiments preferably from about 12 mil to about 13 mil.

The invention is also directed in certain preferred embodiments to a method for providing a quick therapeutic effect to a human selected from a stimulating effect, increased physical endurance, alleviation of temporary fatigue, improved nervous system functions, and a sleep aid, comprising preparing an edible oral film composition comprising a matrix of (i) a thermoplastic food grade material; (ii) a suitable amount of a plasticizer for the thermoplastic food grade material; (iii) an active agent selected from Caffeine, one or more B Vitamins, Vitamin E, and combinations of any of the foregoing; and (iv) one or more flavoring agents, such that the oral strip have a thickness from about 2 mil to about 25 mil; cutting the film composition into suitably sized edible oral strips containing effective amounts of the active agent(s) and the flavoring agent(s); and packaging the edible oral strip. In certain embodiments, a plurality of the edible oral strip compositions are packaged in a dispenser such that a human can remove an edible strip composition from the dispenser and place it in the oral cavity, and in other embodiments the oral strip is individually packaged, such that the active agent(s) and flavoring agent(s) are released within about 1 minute, preferably within about 30 seconds, after being placed in the oral cavity.

All percentages provided herein are w/w unless otherwise specified.

For purposes of the present invention, the terms “oral strip” and “edible film” or “film matrix” are interchangeable.

The term “released” as used herein in conjunction with, e.g., the active ingredient in the edible oral film compositions of the present invention means that the active ingredient is solubilized and is not longer contained in the film, and is distinguished from the term “absorbed”. In other words, it is contemplated that the edible oral film compositions of the present invention will release the active ingredient(s), which will then be washed down the gastrointestinal tract where it is absorbed (preferably rapidly).

### DETAILED DESCRIPTION

In certain embodiments, the edible oral strips of the present invention may contain one or more active ingredients that have stimulating effects and increase physical endurance, alleviate temporary fatigue and/or improve nervous system functions. They may be administered for increasing energy, reducing soreness during and after workout routines, and/or increasing mental alertness in a human. This is commonly referred to as providing an “energy boost.” In other embodiments, the edible oral strips of the present invention may contain one or more active ingredients that alone or together may act as a sleep aid in a human.

The edible oral strips of the present invention may be any type of conventional dissolving oral edible strip. In preferred embodiments, the edible oral strip is a quick-dissolve strip. The quick-dissolve strip may be of any shape, such as oblong, square, round, rectangular, etc. The quick-dissolve strip may also have a variety of sizes.

In preferred embodiments of the invention, the oral edible energy strips (film compositions) contain a therapeutically effective amount of at least one active ingredient that has stimulating effects and increase physical endurance, alleviate temporary fatigue and/or improve nervous system functions. The term “therapeutically effective amount” as used in the present specification refers to an amount of the admixture of the active ingredient, when administered to a human via an oral edible strip of the present invention to the gastrointestinal tract, alleviates temporary fatigue and/or provides an increased sensation of energy as can be felt by the human(s), or can act as a sleep aid, within a reasonable lapse of time after the oral administration.

In certain preferred embodiments, the oral edible energy film composition includes caffeine as an active agent. Caffeine is known to be useful as a cardiac stimulant and also is well known as a mental stimulant, due to its affinity for binding to the adenosine receptors in nerve cells. Caffeine is a naturally occurring xanthine alkaloid found in some plants where it acts as a natural pesticide. In humans it may have numerous beneficial effects. The most common use of caffeine as a supplement is as a central nervous system stimulant and performance enhancer, particularly in terms of mood, mental tasks and alertness (Smith A, Sutherland D, Christopher G. Effects of repeated doses of caffeine on mood and performance of alert and fatigued volunteers. J Psychopharmacol. 2005 November; 19(6):620-6). Biochemically, caffeine binds to, but does not activate, adenosine receptors which are normally activated by adenosine to induce sleep (Shi D, Nikodijevic O, Jacobson K A, Daly J W. Chronic caffeine alters the density of adenosine, adrenergic, cholinergic, GABA, and serotonin receptors and calcium channels in mouse brain. Cell Mol Neurobiol. 1993 June; 13(3):247-61) thereby antagonizing the receptors and inducing a more alert state. Furthermore, Caffeine has also been shown to increase the number of adenosine receptors and inhibit adenylate cyclase activity in the rat cerebral cortex (Ramkumar V, Bumgarner J R, Jacobson K A, Stiles G L. Multiple components of the A1 adenosine receptor-adenylate cyclase system are regulated in rat cerebral cortex by chronic caffeine ingestion. J Clin Invest. 1988 July; 82(1):242-7). This leads to an increase in intracellular cyclic adenyl monophosphate (cAMP), an important signaling molecule, which results in increased epinephrine (adrenalin) and norepinephrin (noradrenalin) levels (Thong F S, Derave W, Kiens B, Graham T E, Urso B, Wojtaszewski J F, Hansen B F, Richter E A. Caffeine-induced impairment of insulin action but not insulin signaling in human skeletal muscle is reduced by exercise. Diabetes. 2002 March; 51(3):583-90; Smith A, Brice C, Nash J, Rich N, Nutt D J. Caffeine and central noradrenaline: effects on mood, cognitive performance, eye movements and cardiovascular function. J Psychopharmacol. 2003 September; 17(3):283-92). Levels of cAMP are also increased by the ability of caffeine to inhibit phosphodiesterases that degrade cAMP (Leblanc J, Richard D, Racotta I S. Metabolic and hormone-related responses to caffeine in rats. Pharmacol Res. 1995 September; 32(3):129-33).

A meta-analysis including forty double-blind studies support the use of caffeine to increase physical endurance (Doherty M, Smith P M. Effects of caffeine ingestion on exercise testing: a meta-analysis. Int J Sport Nutr Exerc Metab. 2004 December; 14(6):626-46). The stimulatory effect of caffeine has also been shown to increase the basal metabolic rate (Astrup A, Toubro S, Cannon S, Hein P, Breum L, Madsen J. Caffeine: a double-blind, placebo-controlled study of its thermogenic, metabolic, and cardiovascular effects in healthy volunteers. Am J Clin Nutr. 1990 May; 51(5):759-67; Dulloo A G, Geissler C A, Horton T, Collins A, Miller D S. Normal caffeine consumption: influence on thermogenesis and daily energy expenditure in lean and postobese human volunteers. Am J Clin Nutr. 1989 January; 49(1):44-50) and improve athletic performance (Dodd S L, Herb R A, Powers S K. Caffeine and exercise performance. An update. Sports Med. 1993 January; 15(1): 14-23). In certain preferred embodiments, the caffeine is anhydrous caffeine. Each edible oral strip may contain, e.g., from about 10 mg to about 500 mg of caffeine anhydrous, preferably from about 25 mg to about 200 mg caffeine anhydrous, and most preferably about 50 mg caffeine anhydrous.

In certain preferred embodiments, the oral edible energy film composition also includes includes a therapeutically effective amount of one or more B Vitamins. Non-limiting examples of B Vitamins include Vitamin B5 (pantothenic acid), Vitamin B6 (pyridoxine), Vitamin B7 (biotin), and Vitamin B12 (cyanocobalamin). Other B Vitamins may also be included in the edible oral strips, such as B1 (thiamin or thiamin mononitrate), Vitamin B2, Vitamin B3 (niacinamide), and Vitamin B9 (folic acid). The B Vitamins often work together to deliver a number of health benefits to the body. Together, one or more B Vitamins may work to combat the symptoms and causes of stress, depression and cardiovascular disease. In certain particular embodiments, the one or more B Vitamins are provided in a B Vitamin blend including the following components: Vitamin B5, B6, B7, and B12. The amount of Vitamin B6 included in the edible oral strip of the present invention may be, e.g., from about 0.5 mg to about 25 mg. The amount of Vitamin B5 included in the edible oral strip of the invention may be, e.g., from about 1 mg to about 20 mg, preferably about 5 mg. The amount of Vitamin B7 which is included in the edible oral strip may be from about 10 to about 250 mcg, preferably about 30 mcg. The amount of Vitamin B12 which is included in the edible oral strips of the invention may be, e.g., from about 0.5 mcg to about 20 mcg, and preferably about 6 mcg. Suitable amounts of other B Vitamins are, for example, Vitamin B1 (15 mg), Vitamin B2 (15 mg), Vitamin B3 (50 mg), and folic acid B9 (400 mcg). The Vitamin B can be provided in an amount of 0.25 to 3 percent by weight, 0.5 to 2.5 percent by weight, 0.75 to 2.0 percent by weight, or 1.0 to 1.5 percent by weight of the total weight of the admixture.

Pantothenic Acid (Vitamin B5) is one of eight water-soluble B-Vitamins and is important in the metabolism of carbohydrates. Moreover, Pantothenic Acid is a precursor of coenzyme A which is involved in carbohydrate metabolism. Vitamin B5 is a Vitamin found in numerous cellular reactions in the body that is involved in the breakdown of fat and protein, helps make red blood cells, sex hormones, and adrenal hormones and is used in the manufacture of a neurotransmitter acetylcholine. In addition, Vitamin B5 makes Vitamin B2 made more effective. Vitamin B5 is also part of an enzyme that helps with fat burning (co-enzyme A). Other important functions of Vitamin B5 include its involvement in the manufacturing of melatonin in the body, which is responsible for a good sleep cycle, and its involvement in regulating hormones in the adrenal cortex, which are activated when the body is under stress. Thus, Vitamin B5 may be characterized as an “anti-stress” vitamin. In various embodiments of the present invention which are set forth in greater detail in the example set forth below, the diet supplement includes Calcium Pantothenate. A serving of the diet supplement may include from about 0.0005 g to about 0.004 g of Calcium Pantothenate. The preferred dosage, in a serving of said diet supplement, comprises about 0.0015 g of Calcium Pantothenate.

Vitamin B6, it is a supplement that is involved in a significant number of vital bodily functions. B6 is a co-enzyme involved in the metabolism of protein and carbohydrates, and aids in the formation of blood cells. Vitamin B6 is also involved in the synthesis of neurotransmitters and prostaglandins and is necessary for proper B12 absorption. In addition, Vitamin B6 helps to control stress because it is used in the synthesis of essential neurotransmitters.

Biotin (Vitamin B7) is essential for the activity of many enzyme systems, and plays a strong role in the production of energy from the metabolism of carbohydrates and fats. Biotin is believed to stimulate liver glucokinase activity, increasing insulin production, and enhancing glucose uptake in muscle cells.

Vitamin B12 (Cobalamin) is a cobalt-containing vitamin in the Vitamin B complex. It is obtained in the diet mainly from meat and dairy products. Vitamin B12 is involved in the metabolism of proteins, fats, and carbohydrates and is used to produce succinyl CoA, an intermediary in the Krebs cycle that generates ATP for energy. Vitamin B12 deficiency has been linked to anemia and a number of neuropsychiatric disorders which can be effectively treated with oral supplementation (Oh R, Brown D L. Vitamin B12 deficiency. Am Fam Physician. 2003 Mar. 1; 67(5):979-86).

In certain preferred embodiments, the B Vitamins include one or more B Vitamins selected from Vitamin B6, Vitamin B12, Vitamin B5, and Vitamin B7.

In certain further preferred embodiments of the invention, the oral edible energy strip further includes a therapeutically effective amount of Vitamin E. Vitamin E is the collective name for a group of fat-soluble compounds with distinctive antioxidant activities, which occur naturally in eight chemical forms (alpha-, beta-, gamma-, and delta-tocopherol and alpha-, beta-, gamma-, and delta-tocotrienol). Alpha-tocopherol is the only form that is recognized to meet human requirements. In addition to its activities as an antioxidant, Vitamin E is involved in immune function and, as shown primarily by in vitro studies of cells, cell signaling, regulation of gene expression, and other metabolic processes. Vitamin E also increases the expression of two enzymes that suppress arachidonic acid metabolism, thereby increasing the release of prostacyclin from the endothelium, which, in turn, dilates blood vessels and inhibits platelet aggregation. In certain preferred embodiments, the edible oral strips include Vitamin E in an amount from about 1 mg to about 30 mg. In certain preferred embodiments, the edible oral strips of the invention include Vitamin E Acetate in an amount of about 6.0 mg.

In certain preferred embodiments, the oral edible energy film composition includes a combination of caffeine, Vitamin E and one or more B Vitamins. In certain preferred embodiments, the Vitamin B comprises one or more selected from the group consisting of thiamine, riboflavin, niacin, pantothenic acid, pyrodixine, biotin, cyanocobalamin, folic acid, and reduced forms of folic acid.

In certain preferred embodiments, the oral edible energy film compositions include biotin. Biotin enhances the absorption of macromolecules and charged molecule via the sodium-dependent multivitamin transporter (SMVT). Absorption of biotin in the intestines involves a saturable and Na+-dependent carrier-mediated process that is shared with pantothenic acid and lipoate and many molecules just use this pathway opportunistically for absorption.

Additional ingredients may be incorporated into the oral edible energy strips of the present invention to provide additional therapeutically desired effects, e.g., to provide at least one effect selected from a stimulating effect, an increased physical endurance, promote increased energy, promote mental alertness, alleviate temporary fatigue, improve nervous system functions, and combinations of any of the foregoing, in a human who ingests the edible oral strip. Such additional ingredients include but are not limited to therapeutically effective amounts of enzymes, amino acids, herbs, minerals (e.g., effective amounts of calcium, magnesium and zinc, e.g., for combating stress effects), trace elements, additional vitamins such as Vitamins A or C, and combinations of any of the foregoing. The amount of Vitamin C optionally included in the oral edible strip of the present invention may be from about 50 mg to about 1000 mg, preferably from about 100 mg to about 500 mg. The additional ingredient may include specific antioxidant chemicals, like the polyphenol, resveratrol (from grape seeds or knotweed roots), additional antioxidants such as selenium, or herbs that contain antioxidants such as green tea and jiaogulan. In certain embodiments, the edible oral strips may include calcium and magnesium in therapeutic amounts for the prevention of muscle cramps. In certain preferred embodiments, the calcium to magnesium is in a ratio of 2:1, may include up to, e.g., 1500 mg. calcium and 750 mg magnesium, as well as 400-1000 IU of Vitamin E for prevention of muscle cramps. In such embodiments, potassium salt(s) and/or sodium salt(s) may be incorporated into the edible oral strips of the invention to further aid in the prevention of muscle cramps.

Sleep occupies about one-third of human life and is necessary for mental and physical well-being. Sleep affects mood, behavior and physiology. Sleep and the control of sleep is a complex process involving multiple neuro-chemical pathways and associated brain structures. The regulation of sleep in humans is governed by three processes—each influenced by hormonal and environmental factors: a daily sleep-wake cycle influenced by a circadian rhythm (24 hour cycle) tied to light-dark cycles. Sleep, or the natural periodic suspension of consciousness during which the powers of the body and mind are restored, is an essential component of human life. It has long been clear that sleep is crucial for sustaining normal function and the mental and physical well-being of all animals. It is well accepted that sleep is an opportunity for the human body to get much needed repair. The types of repair during sleep include physical repair, such as the repair of torn muscles, organ cleansing, etc., and psychological repair, such as the laying down of memories, working though anxiety, etc.

The need for sleep is clear. Although the function of sleep is largely unknown, some evidence indicates that sleep is required for learning. In North America, insomnia is estimated to affect a significant portion of the population every year and is associated with health problems and concomitant economic loss to society (Stoller M K. Economic effects of insomnia. Clin Ther. 1994 September-October; 16(5):873-97 Abstract). It is recognized that the impairment of sleep is detrimental to health. In humans, mild sleep deprivation results in indications of impaired immune system function (Irwin M, McClintick J, Costlow C, Fortner M, White J, Gillin J C. Partial night sleep deprivation reduces natural killer and cellular immune responses in humans. FASEB J. 1996 April; 10(5):643-53). Prolonged sleep deprivation is even known to result in death. It has been determined by many that an individual can survive longer without food than one can without sleep; thus indicating the importance of sleep.

It is commonly known and reported that causes of sleep disruptions are multiple and varied (see, for example, When You Cannot Sleep. ABCs of ZZZs, National Sleep Foundation, Washington, D.C. (2003)). One group of causes is psychological factors, with stress being the primary cause of short-term sleeping difficulties. Common triggers include school- or job-related pressures, a family or marriage problem, and a serious illness or death in the family. While most sleep problems disappear when the stressful situation is over, if short-term sleep problems are not managed properly, they can persist long after the original stress has passed. Another common group of causes are lifestyle stressors, including consumption of alcohol or caffeine and other stimulants, exercising close to bedtime, following an irregular morning and nighttime schedule, and working or doing other mentally intense activities right before or after getting into bed. Older adults also have frequent difficulty with sleep. With advanced age, the total amount of sleep needed in a 24 hour period is not reduced, but common sleep disruptors, such as impaired health, pain and the increased use of medications are prevalent. In older adults, sleep-wake cycle disturbances and circadian-based sleep imbalances are also widespread. Reduced endogenous melatonin production that is secondary to the process of aging can cause these sleep disruptions.

Therefore, a composition which improves sleep (e.g., aid in the induction of sleep or ability to remain asleep) would be beneficial to those in need of the same, not only in terms of physical health, but also in terms of emotional health. Furthermore, reinforcement of sleep of adequate quantity and quality positively impacts most aspect of daily life.

The present invention addresses the above unrealized needs by providing compositions and methods of using the compositions for the regulation of sleep and circadian rhythms. The compositions described herein are nutritional supplements containing melatonin and optionally one or more additional ingredients that enhance the effectiveness of melatonin or of the composition for sleep.

Melatonin is a hormone produced by the pineal gland and is derived from the amino acid tryptophan. While possibly being involved in multiple biological processes, melatonin has largely been studied for its involvement in sleep regulation (Karasek M, Winczyk K. Melatonin in humans. J Physiol Pharmacol. 2006 November; 57 Suppl 5:19-39) with respect to the circadian rhythm cycle of an individual. Levels of melatonin cycle in the body based on lighting conditions—i.e. low melatonin levels during the day, higher levels at night. Typically, melatonin levels peak in the middle of the night and diminish thereafter. Melatonin is the principal sleep-regulating hormone in the body. It is normally excreted with day/night cycles. Between the ages of 20 and 70, adults experience about a 37% decline in daily melatonin output. (Zeitzer, J. M., Daniels, J. E., Duffy, J. F., et al.). Melatonin has further been explored as a method to treat sleep disorders such as insomnia and ‘jet lag’ due to its apparent involvement the regulation of circadian rhythms. Melatonin supplementation in humans has been found to be efficacious for treating jet lag (Herxheimer A, Petrie K J. Melatonin for the prevention and treatment of jet lag. Cochrane Database of Systematic Reviews 2002, Issue 2. Art. No.: CD001520. DOI: 10.1002/14651858.CD001520) as well as hastening the onset of sleep (Brzezinski A, Vangel M, Wurtman R, Norrie G, Zhdanova I, Ben-Shushan A, Ford I. 2005. Effects of exogenous melatonin on sleep: a meta-analysis. Sleep Medicine Reviews 9:41-50). Thus, melatonin deficiency is a fundamental deficiency associated with aging. Melatonin supplementation beneficially addresses this deficiency in general, and, more specifically, mitigates the sleep-wake cycle disturbances and circadian-based sleep imbalances associated with the age-related reduction of endogenous melatonin production.

The dosage of melatonin at one time is 0.005 mg/kg body weight to 1000 mg/kg body weight, preferably 0.01 mg/kg body weight to 400 mg/kg body weight, and more preferably 1 mg/kg body weight to 150 mg/kg body weight. The preferred concentration of melatonin in the composition to be administered to a human adult is less than 5 mg by weight. In preferred embodiments of the invention related to strip compositions for aiding sleep, the composition comprises from about 0.1 to about 10 mg melatonin, and more preferably from about 1 mg to about 5 mg melatonin, and most preferably about 3 mg melatonin.

The compositions beneficially and advantageously regulate sleep when administered to an individual and are administered to a human or animal suffering from an irregular sleep or circadian rhythm or are administered in anticipation of the development of such an irregularity.

L-theanine, also known as gamma-glutamethylethylamide and N-ethyl-L-glutamine, is an amino acid found in green tea. It is however distinct from the polyphenols and catechins which are typically associated with the beneficial effects of green tea. While catechins are generally associated with antioxidant activities, L-theanine is associated with anti-stress. In hypertensive rats, L-theanine lowers blood pressure (Yokogoshi H, Kato Y, Sagesaka Y M, Takihara-Matsuura T, Kakuda T, Takeuchi N. Reduction effect of L-theanine on blood pressure and brain 5-hydroxyindoles in spontaneously hypertensive rats. Biosci Biotechnol Biochem. 1995 April; 59(4):615-8 Abstract). Moreover, oral L-theanine administration to humans has additionally been shown to reduce stress (Kimura K, Ozeki M, Juneja L R, Ohira H. L-L-theanine reduces psychological and physiological stress responses. Biol Psychol. 2007 January; 74(1):39-45 Abstract).

In certain preferred embodiments, the oral edible sleep aid strip (film) compositions of the present invention include L-theanine to reduce stress. In certain preferred embodiments, the composition includes from about 1 mg to about 100 mg of L-theanine, and preferably from about 0.1 mg to about 5 mg L-theanine, most preferably about 3 mg.

Chamomiles are plants of the family Compositae that originate in Europe and are among the oldest herbs that have been used since the times of ancient Egypt. The annual German chamomile, the perennial Roman chamomile, etc., are known among chamomiles, and any of these may be used in this invention. The dosage of chamomile at one time is typically regarded to be from about 0.004 mg/kg body weight to about 6000 mg/kg body weight. In certain preferred embodiments, the sleep aid composition includes from about 0.1 mg to about 10 mg of chamomile extract, and preferably from about 1 mg to about 5 mg chamomile extract, most preferably about 3 mg.

In one aspect, the present sleep aid strip compositions include at least one species of Lycium such as, for example, Lycium barbarum, Lycium chinense, or any combination thereof. In this aspect, Lycium barbarum (Goji or Wolfberry) may be used within the composition. Lycium barbarum (L. barbarum) is a solanaceous defoliated shrubbery and the fruit (goji) has been a commonly prescribed traditional medicine in Asian countries for over 2,500 years. Modern studies indicate that L. barbarum and its main active constituents, L. barbarum polysaccharides (LBP) possess a range of biological effects, such as significantly increasing metabolic rate and body weight reduction in mice and rats. Also observed effects include significant clinical improvements in general well-being, including energy levels, sleep quality, glucose control in diabetics, glaucoma, anti-oxidant properties, anti-aging, neuroprotection, anti-fatigue/endurance, immunomodulation, anti-tumor activity and cytoprotection. In certain preferred embodiments, the composition includes from about 0.1 mg to about 100 mg of goji berry, and preferably from about 1 mg to about 5 mg goji berry, most preferably about 3 mg.

One important benefit of the edible film compositions of the present invention is that they can provide the desired benefits, e.g., stimulating effects and increase physical endurance, alleviate temporary fatigue and/or improved nervous system functions, or act as a sleep aid, while not increasing body fluid content. This is valuable for persons who need the energy boost but at the same time are concerned about increased diuresis from fluid load typically encountered with liquid energy compositions. This makes the present edible film compositions very practical for, e.g., truck drivers and athletes who cannot afford the inconvenience of having to take a bathroom break. An oral edible strip is particularly important in offsetting the effect of caffeine, which is a diuretic.

The compositions of the invention will have a thickness suitable for its intended application. The appropriate thickness will allow the composition to adequately release the flavoring and/or coloring ingredients. Also the thickness will be suitable for manufacturing, cutting or stamping, packaging and handling. The composition thickness may be tested by any procedure known to one of ordinary skill in manufacturing. The oral strips of the invention are preferably from about 2 mil to about 25 mil, and in certain embodiments preferably from about 12 mil to about 13 mil.

Thick compositions, e.g., films, will have a negative impact on the drying process because removing the water from the wet solution will take a longer period of time for a thick composition when compared to a thin composition made from the same formula.

The oral strip compositions of the invention may include further ingredients such as release aids, processing aids, colorants, etc. The edible meltable film matrix compositions in accordance with the present invention may include one or more processing aids such as texture adjusting agents, cooling agents, stabilizing agents, emulsifying agents, thickening agents, binding agents, sweeteners, mixtures thereof, and the like.

By way of example, and not limitation, the film layer can be produced using a highly water-soluble polymer(s) comprising a natural and/or synthetic water-soluble polymer. The polymer preferably has good film moldability, produces a soft flexible film, and is safe for human consumption. Such polymer(s) can be a water-soluble cellulose derivative like hypromellose (hydroxypropylmethylcellulose), hydroxypropyl cellulose (HPC), methyl cellulose, hydroxypropyl alkylcellulose, carboxymethyl cellulose or the salt of carboxymethyl cellulose. The film layer may also be produced using poly vinyl alcohol, poly vinyl pyrrolidone, polyalkylene glycol, hydroxy propyl starch, alginic acid or its salt, poly-saccharide or its derivatives such as trangacanth, gum gelatin, collagen, denatured gelatin, and collagen treated with succinic acid, anhydrous phthalic acid, pullulan, maltodextrin, pectin, alginates, carrageenan, guar gum, exudate gums (arabic, ghatti, karaya, tragacanth), extract gums (β-glucans, inulins, konjac, larch), seed gums (locust bean, guar, pysllium, quince, fenugreek, tara), pectins (high methoxy-, low methoxy-, amidated), microbial gums (xanthan, curdlan, pullulan, gellan, scleroglucan, welan, rhamsan), modified celluloses (methylcellulose, hydroxypropylcellulose, hydroxypropylmethyl cellulose, sodium carboxymethyl cellulose), seaweed hydrocolloid extracts (sodium alginate, propyleneglycol alginate, modified alginate, ammonium alginate, alginic acid, carageenans (iota, ι, kappa, κ, lambda, λ)), dextrins, dextran, hydrogenated starch hydrolyzates, polydextrose, agar, modified agar, gelatins (both type A and B, hydrolyzed gelatin, modified gelatin), milk proteins (whole milk protein, sodium caseinate, calcium caseinate, whey proteins, albumins, lactoglobulins), pregelatinized starches, seed proteins (from soy, sunflower, cottonseed, peanut), cereal proteins (wheat, corn, oat, rice), fractionated proteins, hydrolyzed proteins, chitosan, and modified chitosan, other gelatins, mixtures of any of the foregoing, and the like.

In certain preferred embodiments, the thermoplastic food grade material which may be used in the edible oral strips of the invention include, but are not limited to, hydroxypropylmethyl cellulose, hydroxyethyl cellulose, hydroxypropyl cellulose, hydroxyalkyl methyl cellulose, polyvinyl pyrrolidone, carboxymethyl cellulose, polyvinyl alcohol, sodium alginate, polyethylene glycol, xanthan gum, tragacanth gum, guar gum, pullulan, acacia gum, arabic gum, mixtures of any of the foregoing, and the like. In certain preferred embodiments, the thermoplastic food grade material which is incorporated into the oral strips of the invention comprises a mixture of at least two hydroxypropylcelluloses having differing molecular weights. An example of a combination of such materials is hydroxypropylcellulose having a molecular weight of about 95,000 (e.g., Klucel®-L) and a hydroxypropylcellulose having a molecular weight of about 40,000 (e.g., Klucel®-ELF).

The oral strips of the invention may also comprise poorly water-soluble cellulose derivatives including ethyl cellulose, cellulose acetate and butyl cellulose; shellac; higher fatty acids including steric acid and palmitic acid, an acrylic acid copolymer or its sodium, potassium or ammonium salt.

Additional agents that may be incorporated into the edible oral strips of the invention include breath freshening compounds like menthol, peppermint oil, and the like; other flavors or fragrances commonly used for oral hygiene; and/or actives used for dental and/or oral cleansing like quarternary ammonium bases. Other useful active ingredients include zinc oxide, local anesthetics, The effect of flavors may be enhanced using flavor enhancers like tartaric acid, citric acid, vanillin, or the like. Colorants which may optionally be mixed in the film must be safe in terms of toxicity and should be accepted by the Food and Drug Administration for use in cosmetics.

Plasticizers which may be included in the edible oral strips of the present invention include, but are not limited to: low molecular weight polyols (e.g., glycerin, propylene glycol); polyethylene glycols with molecular weight less than 1,000 daltons; polypropylene glycols with molecular weight of 200 daltons or less; glycol esters (e.g., propylene glycol monethyl ether); esters (e.g., sorbitol lactate, ethyl glycol); amines (e.g. triethanolamine); and sugars (e.g. sorbitol, sucrose), glycerin, propylene glycol, polyethylene glycol, triacetin, diacetin, triethylcitrate, a polyol, a modified polyol or food grade materialic alcohol or polyol or urea, triacetin, monoacetin, diacetin, glycerol, diglycerol, propylene glycol, triethylene glycol, erythritol, sorbitol, mannitol, maltitol, hydrogenated starch syrup, polyvinyl alcohol, polyethylene oxides, polyethylene glycol, urea, mixtures thereof and the like.

In certain embodiments, the plasticizer is employed in an amount sufficient to impart flexibility to the resulting film sheet. In certain embodiments, the plasticizer is preferably present in an amount from about 0-45% w/w.

Detackifiers which may be included in the edible oral strips of the present invention include but are not limited to: water insoluble polymers (e.g., cellulose acetate phthalate, polymethacrylate); lipids and fatty acids (e.g., carnauba wax, cetyl alcohol); inorganic diluents (e.g., calcium carbonate, talc); disintegrants (e.g., crosarmellose sodium, starch, microcrystalline cellulose); and, sugars (e.g., mannitol, xylitol, maltitol, lactose).

Taste modifying agents which may be included in the edible oral strips of the present invention include but are not limited to: flavoring agents, sweetening agents and taste masking agents. Examples of taste modifying agents suitable for use with the present invention include: the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, licorice, thymol, eucalyptol, honey, pineapple, peach, kiwi, papaya, mango, coconut, apple, coffee, plum, watermelon, nuts, durian and green tea. Encapsulation of the active agent or combination of active agents may also be utilized to achieve taste masking of active agents that are bitter.

Buffering agents may also be optionally incorporated into the edible oral strips of the present invention, and include acidulants and alkalizing agents. Examples of buffering agents suitable for use with the present invention include but are not limited to: citric acid, fumaric acid, lactic acid, tartaric acid, malic acid, as well as sodium citrate, sodium bicarbonate and carbonate, and sodium or potassium phosphate.

Coloring agents suitable for use in the edible oral strips of the present invention include, but are not limited to: FD & C coloring agents, natural coloring agents, and natural juice concentrates, pigments such as titanium oxide, silicon dioxide and zinc oxide.

Preservatives suitable for use in the edible films of the present invention include but are not limited to: anti-microbial agents and non-organic compounds. Examples of preservatives suitable for use with the present invention include sodium benzoate, parabens and derivatives, sorbic acid and its salts, propionic acids and its salts, sulfur dioxide and sulfites, acetic acid and acetates, nitrites and nitrates.

Surfactants which may be included in the edible oral films of the present invention include, but are not limited to, mono- and di-glycerides of fatty acids and polyoxyethylene sorbitol esters, such as Atmos 300 and Polysorbate 80, pluronic acid, sodium lauryl sulfate, and the like. In certain embodiments, the surfactant can be added in amounts ranging from 0 to about 5 wt % of the film. Sweetening agents for use in the films prepared in accordance with the present invention include both natural and artificial sweeteners. Suitable sweeteners include, but are not limited to, water-soluble sweetening agents such as monosaccharides, disaccharides and polysaccharides such as xylose, xylitol, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like; dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5, dihydrophenyl-glycine, L-aspartyl-2,5-dihydro-L-phenylalanine, L-aspartyl-L-(1-cyclohexyen)-alanine, 1,6-dichloro-1,6-dideoxy-β-D-fructofuranosyl-4-chloro-4-deoxy-α-D-galactopyranoside (sucralose), mixtures thereof, and the like. In certain embodiments, an effective amount of sweetener is utilized to provide the level of sweetness desired to impart to a food substrate, and this amount will vary with the sweetener selected.

As discussed previously, various optional ingredients such as conventionally used in the art, may be included in the compositions of the present invention. For example, colorings, sweeteners (such as sucrose and sucralose), food acids (such as malic acid and citric acid), salts (such as calcium citrate and calcium chloride), fragrances, diluents, flavor maskers, flavor enhancers, plasticizers, fillers, preservatives, anti-oxidants, pH modifying agents, thickening agents, binding agents, cooling agents, emulsifying agents, stabilizers, lubricants, and the like may be employed herein if desired.

The edible oral strips of the present invention may be prepared, e.g., as matrix film compositions. The edible oral strips of the present invention may be manufactured utilizing various techniques well known to those having ordinary skill in the art.

In certain embodiments, the compositions of the present invention may be manufactured by any suitable mixing process. For example, in certain embodiments, all of the ingredients except water are mixed together to form a granular powder. Thereafter, the water is slowly added, and mixed well. In other embodiments, the process may be reversed. The resultant mixture may then be cast, e.g., @ 130 mils and then dried (e.g., air dried) to obtain a film having a desired thickness (e.g., from about 10 mil to about 50 mil).

The mixing methods described above may be carried out utilizing various mixing apparatuses. For example, in certain embodiments of the invention, a suitable apparatus for the preparation of the matrix compositions described herein includes a storage vessel which contains a solution to be formed into film strips; an endless belt supported by a plurality of rotating rolls which carries a cast layer of solution from the storage vessel beneath a heated dryer or heated air for drying, such that the solution dries and forms a film; thereafter the film is stripped from the endless belt by a film stripper. In such systems, for example, the solution is continuously cast on the endless belt allowing for the continuous production of film.

The edible oral strips of the present invention may also be prepared by forming matrix film compositions utilizing an extrusion process. For example, in certain embodiments, the matrix film compositions of the present invention are manufactured by extruding a plasticized mixture of thermoplastic food grade material and a flavoring component through an extruder pre-heated to a temperature from about 100° F. to about 250° F. to form an extrudate, and shaping the extrudate into a desired shape for further processing.

The processing conditions and amounts of ingredients (e.g., dry and liquid components) may be adjusted to optimize the formation of suitable film matrixes.

The ratio of thermoplastic food grade material (film forming food grade material) to water is such that acceptable properties, e.g., film properties, are obtained during or after the manufacturing process.

In certain other embodiments, the invention is directed to a manufacturing process where at least one of the wet ingredients is sprayed onto the dry ingredients prior to introduction into the extruder. In certain embodiments, water may be added to the wet ingredients prior to the wet ingredients being sprayed onto the dry ingredients. In other embodiments, water may be added to a mixture of wet and dry ingredients prior to the extrusion process.

In certain embodiments, the films are cooled prior to further processing. In certain other embodiments, a chilling table may be used to cool the films. In still other embodiments, the shaping device, e.g., drum dryer is cooled.

In any event, the edible oral strips of the present invention may be manufactured in any effective manner known to those skilled in the art. For example, U.S. Patent Application Nos. 20010022964, 20020131990 and 20020019447 and U.S. Pat. Nos. 6,419,903, 3,931,146, 5,411,945, 6,010,716, 5,629,003, 5,948,430, 6,177,096, 6,284,264, 5,700,478, 6,449,925, 4,072,551, 4,083,741, all of which are incorporated herein by reference as if fully set forth herein, describe methods for making edible films. These, and other methods known in the art, or described herein, may be used in accordance with the present invention.

The edible film compositions are then preferably cut into suitably sized oral strips which contain, e.g., effective amounts of the active agent(s) and the flavor(s). The oral strips may be packaged in any suitable manner for commercial sale.

### DETAILED DESCRIPTION OF THE PREFERRED EMBODIMENTS

Features of the invention will become apparent in the course of the following descriptions of exemplary embodiments which are given for illustration of the invention and are not intended to be limiting thereof.

In the following examples, and throughout this specification, all parts and percentages are by weight, unless expressly stated to be otherwise. Where the solids content of a dispersion or solution is reported, it expresses the weight of solids based on the total weight of the dispersion or solution, respectively. Where a molecular weight is specified, it is the molecular weight range ascribed to the product by the commercial supplier, which is identified. Generally this is believed to be weight average molecular weight.

### EXAMPLE 1

In Example 1, an oral film for increasing energy and/or mental alertness in an individual, e.g., a human, is provided. The oral film is provided with a cinnamon flavor. The film is prepared containing the following active ingredients:

Caffeine—50.0 mg; Vitamin E Acetate—6.0 mg; Vitamin B5—5.0 mg; Vitamin B6—2.0 mg; Biotin—30.0 mcg; Vitamin B12—6.0 mcg.

The film is prepared in accordance with the following general procedure: A batch sheet containing a list of all the ingredients set forth in Table 1 below, the exact amounts to be weighed, the order that they are to be used, and the process instructions is generated, e.g., from a Chemical Management system (CMS). The ingredients are weighed on a scale and the CMS generates a bar code label for each raw material. CMS will not generate a label if the amount weighed is incorrect, or out of sequence, or the ingredient has expired, or is in quarantine.

The ingredients are blended in a stainless steel vessel with a homogenizer, side sweep and paddle mixers. When the blending process is complete, a vacuum is pulled to remove bubbles. The completed blend is transferred to a holding tank and kept agitated.

Thereafter, the blend is pumped onto a silicone coated release liner and coated to a target wet coat thickness using knife-over-roll, and passed through an air impinged drying tunnel at 212-245° F. The dry coated film exits the drying tunnel and is slit into spools for die cutting and packaging.

The film of Example 1 including the ingredients of Table 1 has a cinnamon flavor. The film is cut into suitably sized oral edible strips, and then can be suitably packaged. Each oral strip may be consumed as needed throughout the day. Each of the strips includes the ingredients set forth in Table 1 below:

TABLE 1

Vitamin E Acetate 6 mg
Vitamin B6 2 mg
Vitamin B12 6 mcg
Biotin 30 mcg
Pantothenic Acid (Vitamin B5) 5 mg
Caffeine Anhydrous 50 mg
Maltodextrin
Pullulan
Hypromellose
Glycerin
Natural/Artificial Flavors
Propylene Glycol and Natural Flavors
Ethylcellulose
Sucralose
Polysorbate 80
Xylitol
Artifical Flavoring(N,2,3---Trimethyl---2---isopropyl Butanamide)
FD&C Red #40

### EXAMPLE 2

In Example 2, an oral film for providing an energy boost to the user is prepared. The oral film is provided with a berry flavor. The film is prepared containing the following active ingredients: Caffeine—50.0 mg; Vitamin E Acetate—6.0 mg; Vitamin B5—5.0 mg; Vitamin B6—2.0 mg; Biotin—30.0 mcg; Vitamin B12—6.0 mcg.

The film is prepared in accordance with the following general procedure:

A batch sheet containing a list of all the ingredients set forth in Table 2 below, the exact amounts to be weighed, the order that they are to be used, and the process instructions is generated, e.g., from a Chemical Management system (CMS). The ingredients are weighed on a scale and the CMS generates a bar code label for each raw material. CMS will not generate a label if the amount weighed in incorrect, or out of sequence, or the ingredient has expired, or is in quarantine.

The ingredients are blended in a stainless steel vessel with a homogenizer, side weep and paddle mixers. When the blending process is complete, a vacuum is pulled to remove bubbles. The completed blend is transferred to a holding tank and kept agitated.

Thereafter, the blend is pumped onto a silicone coated release liner and coated to a target wet coat thickness using knife-over-roll, and passed through an air impinged drying tunnel at 212-245° F. The dry coated film exits the drying tunnel and is slit into spools for die cutting and packaging.

The ingredients used in the film of Example 2 are set forth in Table 2. The film has a berry flavor.

TABLE 2

Vitamin E Acetate 6 mg
Vitamin B6 2 mg
Vitamin B12 6 mcg
Biotin 30 mcg
Pantothenic Acid (Vitamin B5) 5 mg
Caffeine Anhydrous 50 mg
Maltodextrin
Pullulan
Hypromellose
Glycerin
Natural/Artificial Flavors
Propylene Glycol and Natural Flavors
Xylitol
Ethylcellulose
Sucralose
Polysorbate 80
Artifical Flavoring(N,2,3---Trimethyl-2-isopropyl Butanamide)
Citric Acid
FD&C Red #40
FD&C Blue #1

The film is cut into suitably sized oral edible strips, and then can be suitably packaged. Each oral strip may be consumed as needed throughout the day.

### EXAMPLE 3

In Example 3, an oral film for providing an energy boost to the user is prepared in accordance with the methods set forth in Examples 1 and 2. The oral film is provided with a mint flavor. It contains the following ingredients: Active Ingredients: Vitamin E-6 mg, Vitamin B6-2 mg, Vitamin B12-6mcg, Biotin-30mcg, Pantothenic Acid (B5)-5 mg, Caffeine Anhydrous-50 mg. Inactive Ingredients: Pullulan, Maltodextrin, Hypromellose, Glycerin, Xylitol, Natural/Artificial Flavor(s), Menthol, Ethyl Cellulose, Propylene Glycol and Natural Flavors, Polysorbate 80, Sucralose, Artificial Flavoring (N,2,3-Trimethyl-2-isopropyl Butanamide), and FD&C Blue#1.

### EXAMPLE 4

In Example 4, an oral film for providing sleep aid to the user is prepared in accordance with the methods set forth in Examples 1 and 2. It contains the following ingredients:

Active Ingredients: Melatonin-3 mg, l-L-theanine-3 mg, Goji Berry Extract-1 mg, Chamomile Extract-1 mg

Inactive Ingredients: Hypromellose, Maltodextrin, Glycerin, Oat Fiber, Natural Peppermint Flavor, Gum Arabic, Sucralose, Polysorbate 80.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An edible energy oral strip composition, comprising an active agent selected from Caffeine, one or more B Vitamins, Vitamin E, and combinations of any of the foregoing, the active agent(s) being included in the edible oral strip composition in a therapeutically effective amount to provide at least one effect selected from a stimulating effect, an increased physical endurance, promote increased energy, promote mental alertness, alleviate temporary fatigue, improve nervous system functions, and combinations of any of the foregoing, the edible oral strip further comprising one or more flavoring agents, the composition quickly releasing the active agent(s) from the strip within about 1 to about 30 minutes after being placed in the oral cavity of a human.

2. The edible oral strip composition of claim 1 which includes caffeine as an active agent.

3. The edible oral strip composition of claim 1, wherein the edible oral strip composition includes a combination of Caffeine, Vitamin E and one or more B Vitamins.

4. The edible oral strip composition of claim 1, wherein the B Vitamins include one or more B Vitamins selected from Vitamin B6, Vitamin B12, Vitamin B5, and Vitamin B7.

5. The edible oral strip composition of claim 3, which includes a B Vitamin selected from Vitamin B6, Vitamin B12, Vitamin B5, Vitamin B7, and combinations of any of the foregoing.

6. The edible oral strip composition of claim 1, which further comprises a matrix of (i) at least one thermoplastic food grade material; (ii) a suitable amount of a plasticizer for the thermoplastic food grade material; and (iii) an effective amount of a flavoring agent incorporated therein.

7. The edible oral strip composition of claim 1, which has a thickness from about 2 mil to about 25 mil.

8. The edible oral strip composition of claim 1, which releases the active agent(s) within about 1 minute when placed in the oral cavity of a human.

9. The oral strip of claim 1, wherein the flavoring comprises a sweetener.

10. The oral strip of claim 7, which has a thickness from about 12 mil to about 13 mil.

11. An edible oral strip composition for providing at least one effect selected from a stimulating effect, increased physical endurance, alleviation of temporary fatigue, and improved nervous system functions when placed in the oral cavity of a human, comprising a matrix of (i) a thermoplastic food grade material; (ii) a suitable amount of a plasticizer for the thermoplastic food grade material; (iii) an effective amount of an active agent selected from Caffeine, one or more B Vitamins, Vitamin E, and combinations of any of the foregoing; and (iv) an effective amount of a flavoring agent, the oral strip having a thickness from about 2 mil to about 25 mil and releasing the active agent(s) and flavoring agent(s) within about 1 minute when placed in the oral cavity of a human.

12. The edible oral strip composition of claim 11, wherein the edible oral strip composition includes a combination of Caffeine, Vitamin E and one or more B Vitamins.

13. The edible oral strip composition of claim 12, wherein the B Vitamins include one or more B Vitamins selected from Vitamin B6, Vitamin B12, Vitamin B5, Vitamin B7, and combinations of any of the foregoing.

14. The edible oral strip composition of claim 13, which includes about 50 mg Caffeine anhydrous, about 5 mg Vitamin B5, about 2 mg Vitamin B6, about 30 mcg Vitamin B7 and about 6 mcg Vitamin B12, and about 6 mg Vitamin E acetate.

15. The edible oral strip composition of claim 14, which has a fruit flavor.

16. The edible oral strip composition of claim 14, which has a mint flavor.

17. The edible oral strip composition of claim 14, which has a cinnamon flavor.

18. The edible oral strip compositon of claim 14, wherein the thermoplastic food grade material comprises a mixture of hydroxypropylmethylcellulose and pullulan.

19. The oral strip of claim 17, further comprising a sweetener.

20. A method for providing a quick therapeutic effect to a human selected from stimulating effect, increased physical endurance, alleviation of temporary fatigue, and improved nervous system functions, comprising
preparing a edible oral film composition comprising a matrix of (i) a thermoplastic food grade material; (ii) a suitable amount of a plasticizer for the thermoplastic food grade material; (iii) an active agent selected from Caffeine, one or more B Vitamins, Vitamin E, and combinations of any of the foregoing; and (iv) one or more flavoring agents, such that the oral strip have a thickness from about 2 mil to about 25 mil, such that when a human places it in the oral cavity, such that the active agent(s) and flavoring agent(s) are released within about 1 minute after being placed in the oral cavity;
cutting the film composition into suitably sized edible oral strips containing effective amounts of the active agent(s) and the flavoring agent(s); and
packaging the edible oral strip composition.

21. An edible sleep aid oral strip composition, comprising an active agent selected from melatonin, L-theanine, goji berry, chamomile, and combinations of any of the foregoing, the active agent(s) being included in the edible oral strip composition in a therapeutically effective amount to provide sleep aid, the edible oral strip further comprising one or more flavoring agents, the composition quickly releasing the active agent(s) from the strip within about 1 to about 30 minutes after being placed in the oral cavity of a human.

22. The edible oral strip of claim 21, which comprises from about 1 mg to about 5 mg melatonin.

23. The edible oral strip of claim 21, which comprises from about 1 mg to about 5 mg L-theanine.

24. The edible oral strip of claim 21, which comprises from about 1 mg to about 5 mg goji berry.

25. The edible oral strip of claim 21, which comprises from about 1 mg to about 5 mg chamomile.

26. The edible oral strip of claim 21, which comprises about 3 mg melatonin, about 3 mg L-theanine, about 1 mg goji berry, and about 1 mg chamomile.

27. An edible oral strip composition for providing sleep aid when placed in the oral cavity of a human, comprising a matrix of (i) a thermoplastic food grade material; (ii) a suitable amount of a plasticizer for the thermoplastic food grade material; (iii) an effective amount of an active agent selected from melatonin, L-theanine, goji berry, chamomile, and combinations of any of the foregoing, to act as a sleep aid; and (iv) an effective amount of a flavoring agent, the oral strip having a thickness from about 2 mil to about 25 mil and releasing the active agent(s) and flavoring agent(s) within about 1 minute when placed in the oral cavity of a human.

28. The edible oral strip composition of claim 27, which has a honey flavor.

29. The edible oral strip composition of claim 27, which has a mint flavor.

30. The edible oral strip composition of claim 27, wherein the thermoplastic food grade material comprises a mixture of hydroxypropylmethylcellulose and pullulan.

31. A method for providing a quick therapeutic effect of sleep aid to a human, comprising
preparing an edible oral film composition comprising a matrix of (i) a thermoplastic food grade material; (ii) a suitable amount of a plasticizer for the thermoplastic food grade material; (iii) an active agent selected from melatonin, L-theanine, goji berry, chamomile, and combinations of any of the foregoing; and (iv) one or more flavoring agents, such that the oral strip have a thickness from about 2 mil to about 25 mil, such that when a human places it in the oral cavity, such that the active agent(s) and flavoring agent(s) are released within about 1 minute after being placed in the oral cavity;
cutting the film composition into suitably sized edible oral strips containing effective amounts of the active agent(s) and the flavoring agent(s); and
packaging the edible oral strip composition.
